(19) United States
(12) Patent Application Publication
(10) Pub. No.: US 2020/0258635 A1
(43) Pub. Date: Aug. 13, 2020

(54) SYSTEM IN A PORTABLE MULTIPLE TYPE DIGITAL PLATFORM TO DETERMINE BODY COMPOSITION, INTERACT WITH THE USER, DISPLAY RESULTS UTILIZING AR & MIXED MEDIA & ENGAGE THE USER VIA TWO WAY AUDIO, TEXT & AI TO MEASURE, TRACK & IMPROVE HEALTH

(71) Applicant: Stayhealthy, Inc., Monrovia, CA (US)

(72) Inventors: David Erceg, South Pasadena, CA (US); Sagi Kormandel, Encino, CA (US); Colin Kenneth Hill, San Dimas, CA (US); Kelly Chu, Los Angeles, CA (US); Andrew Spoone, Los Angeles, CA (US); John R. Collins, Claremont, CA (US)

(21) Appl. No.: 16/788,947

(22) Filed: Feb. 12, 2020

Related U.S. Application Data
(60) Provisional application No. 62/804,912, filed on Feb. 13, 2019.

(51) Int. Cl. G16H 50/30

(52) U.S. Cl. CPC G16H 50/30

(57) ABSTRACT

A system for measuring, tracking, and improving health having a control system with a plurality of downloadable applications, a website with a user interface, connected to the control system, and a personal electronic device adapted to download the downloadable applications. One of the downloadable applications is a body fact index application adapted to calculate and display a body fat percent.

### CROSS REFERENCE TO RELATED APPLICATION

This application claims the benefit of the priority of U.S. Provisional Application No. 62/804,912 filed on Feb. 13, 2019, the contents of this application is hereby incorporated by reference in its entirety.

### BACKGROUND OF THE INVENTION

The present invention is directed to a system in a digital platform to determine an individual's health status, interact with the individual, display results using augmented reality and/or mixed media, and engage the individual via two-way audio, text and AI to measure, track, and improve health.

The personal health of individuals is important in many ways including life span, and quality of life, as well as health costs. While many individuals know they need to improve their health and want to improve their health, for some, there are psychological reasons that prevent improvement. There are many systems available to improve an individual's health. While useful, these systems fail to engage individuals and maintain healthy habits and actions over time. Therefore, a need exists in the art for a system that addresses these deficiencies.

An objective of the present invention is to provide a system adapted to encourage an individual to adopt a healthier lifestyle.

Another objective of the present invention is to provide a system that demonstrates the effect of an unhealthy lifestyle.

These and other objectives will be apparent to others having ordinary skill in the art based upon the following written description, drawings, and claims.

### SUMMARY OF THE INVENTION

A system for measuring, tracking, and improving health includes a control system having a plurality of downloadable applications that is connected to a website having a user interface. A personal electronic device is adapted for downloading the downloadable applications which includes a body fact index application and others designed to encourage a user to engage in caloric burning activities, improve lifestyle choices, and earn rewards.

The body fact index application is adapted to calculate and display a body fat percent. The body fat percent is calculated based upon biometric information input by a user and algorithms that utilize a database that includes data points for different individuals. The body fat percent is preferably displayed on a sliding scale.

Also, upon request, a visual display that shows the effect that would result from the calculated body fat percent is displayed. In one example, the visual display is an AR rendering of an organ.

### BRIEF DESCRIPTION OF THE DRAWINGS

FIG. 1 is a schematic view of a system for measuring, tracking, and improving health; and

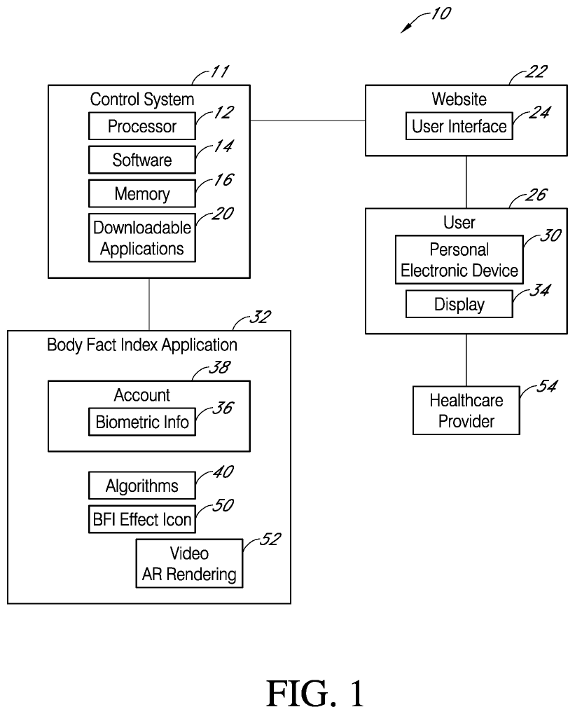

FIG. 2 is a sliding scale for displaying a body fat percent.

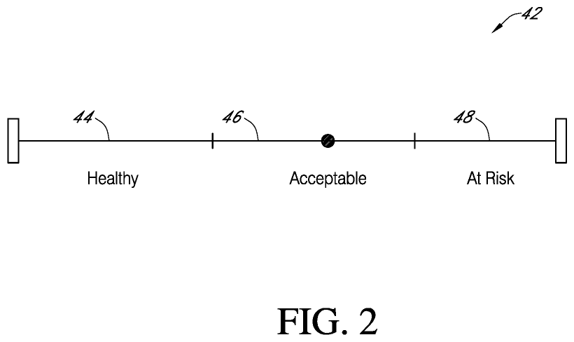

### DETAILED DESCRIPTION OF THE PREFERRED EMBODIMENTS

Referring to the Figures, a system to measure, track, and improve health 10 includes a control system 11, a processor 12, software 14, memory 16 that is either internal or cloud based, and a plurality of downloadable applications 20 preferably related to health. The system is connected to and includes a website, mobile application/platform 22 having a user interface 24.

A user 26 accesses the website 22 through the user interface 24 using a personal electronic device 30 to download one or more of the downloadable applications 20. The personal electronic device 30 is of any type and includes a mobile phone, tablet, personal computer, or the like.

One of the downloadable applications 20 is a body fact index application 32. Once downloaded to the personal electronic device 30, the body fact index application 32 prompts the user 26 with a data screen on a display 34 of the personal electronic device 30 to create an account and input basic biometric information and personal profile information 36. In one embodiment the information is presented as text and images, and in a preferred embodiment it is presented using interactive voice/audio commands. The data screens may be enhanced with augmented reality (AR). Where real time imaging is captured through the electronic device 30. Information input and stored in a user account 38 includes, but is not limited to, the user's name, e-mail, phone number, height, weight, age, race, waist circumference, and other body biometrics.

Once input, the body fact index application 32 estimates a lean body mass and body fat and then using one or more algorithms 40 calculate a body fat percent. The algorithms 40 utilize a large database of biometrics that includes specific data points for different individuals.

Once the body fat percent is calculated, the body fat percent is shown on the display 34 of the user's 26 personal electronic device 30. In one example, the body fat percent is shown on a sliding scale 42 having sections designated for healthy 44, acceptable 46, and at risk 48. The body fact index application 32 then prompts the user 26 by asking if the user 26 wishes to see the effects on the body that result from the body fat percent. The user 26 indicates their choice through a voice command or selection of a button or icon 50 on the display 34. If the user 26 elects to see the effect, the body fact index displays an image still, animated gif and/or animated video 52 of the effect either in 2D, 3D or both. For example, the video 52 may include a ball of fat that starts at a healthy level and grows to a size represented by the user's 26 calculated body fat percent. Alternatively, the video 52 may show the effect of the body fat percent on various organs by showing the organ in a normal state and the organ at the user's 26 body fat percent. The organs may be represented by a realistic AR rendering or a cartoon AR character. One embodiment may also include images or video, in AR, of real subjects or objects (not a rendering or model) using U.S. Pat. Nos. 9,600,939 and 9,972,114 herein incorporated by reference in their entirety as a stand-alone experience or in combination with the rendered objects.

Health tips are also presented by the body fact index application 32 on the display 34 of the personal electronic device 30. The body fact index application 32 also prompts the user 26 by asking if they wish to become a member which permits the user access to other downloadable applications 20 designed to encourage the user 26 to engage in activities that increase calorie burn, improve lifestyle choices, track biometrics, track exercise and/or food logging, plan diet and/or exercise and earn rewards by way of points, tokens, coins, etc. Membership also provides a user 26 the opportunity to purchase downloadable applications 20 related to health. As an example of the downloadable applications 20 available for purchase, one application permits the user 26 to track calories burned through their mobile phone to determine the user's rate of activity and daily, monthly, and yearly average activity which is converted to positive calories stored in a file. In the same application or a different application, a food diary is provided where the user selects food items represented by an AR image that provides calories that are recorded as negative calories in the file. The application calculates the difference between positive and negative calories and presents the difference in fat lost or gained.

The body fact index application 32 and other applications 20, in one example, would have an AR icon, character, screen, spokesman (patent), etc., that pulses, changes colors, asks questions, and responds to user inputs with words and by changing colors and pulse rates. For example, if the user inputs that they ate a double cheeseburger, the AR “character” may turn green, slow its pulse, and have a sad look to demonstrate the user's choice was not healthy. Psychologically, the body fact index application 32 is designed to engage and then gradually change user habits toward a healthier diet, weight loss, and improved muscle tone through exercise with incentives to encourage users to obtain a healthy weight and body fat percentage.

A user 26 can share content with others through social media and the like. The content may be provided by the downloadable application 20 or created by the user 26. For example, information on lung cancer is provided by an application 20 to the user 26 and the user 26 wishes to share the information with a friend or relative who smokes. In another example, a user 26 creates a video with a challenge for others to accept.

Data obtained from the applications 20 is stored for further study, preferably by artificial intelligence, to personalize further interaction with each individual user 26.

The data is stored in the memory 16 of the control system 11, in a cloud based storage, a blockchain storage, or the like. The data is also secured. For example, the data is encrypted on the personal electronic device 30 of the user 26 through a native OS encryption or the like. Also, the data is accessed through a personal verification process. For example, a user 26 maintains a private key in a digital wallet. To access the stored data the user 26 provides the private key which is compared to a stored private key. If they match, access to stored data is provided. A user 26 may share information with a healthcare provider 54 via secured links that follow HIPAA regulations for privacy of the user's medical information.

One of the downloadable applications is directed to weight loss and/or developing healthy eating habits. In one example of this downloadable application 20 a user inputs information about food items they like and/or typically eat. Based upon this input information to control system, after processing the input information, transmits information recommending portion sizes and/or combinations of food items taken from the input information.

From the above discussion and accompanying figures and claims it will be appreciated that the system to measure, track, and improve health 10 offers many advantages over the prior art. It will be appreciated further by those skilled in the art that other various modifications could be made to the device without parting from the spirit and scope of this invention. All such modifications and changes fall within the scope of the claims and are intended to be covered thereby. It should be understood that the examples and embodiments described herein are for illustrative purposes only and that various modifications or changes in the light thereof will be suggested to persons skilled in the art and are to be included in the spirit and purview of this application.

What is claimed is:

1. A system for measuring, tracking, and improving health, comprising:
a control system having a processor, software, memory, and downloadable applications;
a website having a user interface connected to the control system;
a personal electronic device adapted to download the downloadable applications from the control system;
wherein one of the downloadable applications is a body fact index application adapted to calculate and display a body fat percent.

2. The system of claim 1 wherein the body index percent is calculated based upon biometric information input by a user and algorithms that utilize a database that includes data points for different individuals.

3. The system of claim 1 wherein the body fat percent is displayed as a sliding scale having sections designated as healthy, acceptable, and at risk.

4. The system of claim 1 wherein the system is adapted to permit a user to request a visual display of an effect that would result from the calculated body fat percent.

5. The system of claim 4 wherein the visual display is an AR rendering of an organ.

6. The system of claim 1 wherein health tips are displayed on the personal electronic device.

7. The system of claim 1 wherein the downloadable applications are designed to encourage a user to engage in caloric burn activities, improve lifestyle choices, and earn rewards.

8. The system of claim 1 wherein the body fact index application displays an AR character that pulses, changes colors, ask questions and responds to user inputs with words, changing colors, and changing pulse rates.

9. The system of claim 1 wherein data obtained from the downloadable applications is stored for further study by artificial intelligence to personalize further interaction with a user.

10. The system of claim 9 wherein the stored data is shared with a healthcare provider.

11. The system of claim 1 wherein one of the downloadable applications is adapted to promote healthy eating and weight loss.

12. The system of claim 1 wherein the downloadable applications include applications for increasing caloric burn, improving lifestyle choices, calorie tracking, exercise tracking, food logging and food and exercise planning.
